(19) United States
(12) Patent Application Publication
(10) Pub. No.: US 2022/0026417 A1
(43) Pub. Date: Jan. 27, 2022

(54) METHOD OF SIMULTANEOUSLY DIAGNOSING ACTIVE TUBERCULOSIS AND LATENT TUBERCULOSIS INFECTION USING HUMAN WHOLE BLOOD SAMPLE-DERIVED BIOMARKER

(71) Applicant: Industry-University Cooperation Foundation, Catholic University of Pusan, Busan (KR)

(72) Inventors: Sunghyun KIM, Busan (KR); Jungho KIM, Busan (KR); Sung-Bae PARK, Busan (KR); Heechul PARK, Busan (KR); Jun Seong KIM, Busan (KR); Yun-Jeong KANG, Busan (KR)

(73) Assignee: Industry-University Cooperation Foundation, Catholic University of Pusan, Busan (KR)

(21) Appl. No.: 17/153,676

(22) Filed: Jan. 20, 2021

Related U.S. Application Data
(60) Provisional application No. 63/129,234, filed on Dec. 22, 2020.

(30) Foreign Application Priority Data
Jul. 21, 2020 (KR) 10-2020-0090322

(51) Int. Cl. G01N 33/50; G01N 33/68

(52) U.S. Cl. CPC G01N 33/5094; G01N 2496/05; G01N 2800/12; G01N 33/6893

(57) ABSTRACT

The present invention relates to a method of simultaneously diagnosing active tuberculosis and latent tuberculosis infection (LTBI) using one or more biomarkers selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate, or a combination thereof. The present invention may provide a diagnostic method for simultaneously differentiating active tuberculosis and LTBI without a separate additional test on a patient diagnosed as positive by a conventional tuberculosis infection assay such as a tuberculin skin test (TST) or an interferon-γ release assay (IGRA).

### CROSS-REFERENCE TO RELATED APPLICATION

This application claims the benefit of priorities to Korean Patent Application No. 10-2020-0090322 filed on Jul. 21, 2020, and U.S. Provisional Patent Application No. 63/129,234 filed on Dec. 22, 2020. The disclosures of which are incorporated herein by reference in their entirety.

### BACKGROUND

### 1. Field of the Invention

The present invention relates to a method of simultaneously diagnosing active tuberculosis and latent tuberculosis infection using a human whole blood sample-derived biomarker, and more particularly, to a diagnostic method for simultaneously differentiating active tuberculosis and latent tuberculosis infection using a specific biomarker and indicator isolated from a human whole blood (blood) sample as a target.

### 2. Discussion of Related Art

Tuberculosis (TB) caused by Mycobacterium tuberculosis (MTB) infection is a major public health issue worldwide, and one of the most serious diseases with high infection rates and mortality rates. In 2018, approximately 10 million people had tuberculosis, and approximately 1.2 million people died of MTB infection. In addition, the number of multi-drug resistant (MDR)-TB and extensively drug-resistant (XDR)-TB cases increased worldwide. Approximately 5% of people infected by MTB suffered from active tuberculosis for 2 to 5 years, and the other 95% have latent tuberculosis infection (LTBI). LTBI generally exhibits no clinical symptoms of active tuberculosis such as fever, chills, night fever, weight loss, coughing, hemolysis and abnormalities found on chest X-ray examination.

Currently, approximately a third of the world's population has LTBI, and approximately 10% of them develop active tuberculosis during their lifetime. It is known that co-infection of MTB and human immuno-deficient virus (HIV) promotes a serious condition, and people with HIV are easily infected by Mycobacteria. In 2018, among the HIV infected people worldwide, 251,000 TB-related deaths were reported, which accounts for 8.6% of HIV-positive tuberculosis patients. In addition to HIV, the risk of developing active tuberculosis from LTBI is higher in children, patients with diabetes, malignant tumors and kidney diseases, and immunosuppressed patients with long-term use of immunosuppressants. A chronic kidney disease with a high urea level is an important factor in immune suppression, and disrupts the immune mechanism of the body. These patients are susceptible to an opportunistic infection such as tuberculosis.

Recent LTBI diagnostic methods use a chest X-ray (CXR) examination, a physical check-up, TB exposure and medical history data, a tuberculin skin test (TST) or an interferon-gamma (IFN-γ) release assay (IGRA) to diagnose LTBI. The TST is based on intradermal injection of a whole MTB antigen and the subsequent confirmation of delayed hypersensitivity at the injection site. However, since the TST has low sensitivity to Bacillus Calmette-Guirin (BCG) vaccination, non-tuberculosis mycobacteria (NTM) infection and immunosuppression, a false-negative or false-positive result is shown. The IGRA is a whole blood test using a cell-mediated immune response, and a more rapid and sensitive test method than the TST measuring IFN-γ released from a CD4⁺ T-lymphocyte exposed to the MTB antigens. However, the TST and IGRA tests do not differentiate active tuberculosis from LTBI. Therefore, these disease conditions have to be identified and quickly tested. Recently, QuantiFERON-TB plus (Qiagen) is equipped with a QFT-Plus tube containing a new peptide inducing IFN-γ production in CD4⁺ and CD8⁺ T-lymphocytes.

Various studies for differentiating LTBI from active TB were conducted through genes differentially expressed using an MTB antigen-specific cytokine, a reverse transcriptase chain reaction (RT-PCR) assay and a monocyte chemoattractant protein-1 (MCP-1).

### PRIOR ART DOCUMENT

### Non-Patent Document

(Non-Patent Document 0001) (Thesis) Campbell J, Krot J, Cook V, Marra F. (2015). A systematic review on TST and IGRA tests used for diagnosis of LTBI in immigrants, Molecular Diagnosis & Therapy, 19(1):9-24

### SUMMARY OF THE INVENTION

The present invention is provided to solve the above-described problems, and provide a diagnostic method for simultaneously differentiating active tuberculosis and latent tuberculosis infection (LTBI) by a simple method without an additional test.

One aspect of the present invention provides a method of simultaneously diagnosing active tuberculosis and LTBI using one or more selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate, or a combination thereof as a biomarker.

In addition, the method of simultaneously diagnosing active tuberculosis and LTBI according to the present invention may use one or more selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate of a specimen isolated from a human whole blood (blood) sample, or a combination thereof as a biomarker.

Another aspect of the present invention provides a method of simultaneously diagnosing active tuberculosis and LTBI, which includes: providing a whole blood (blood) sample of a patient diagnosed as positive in a tuberculin skin test (TST) or an interferon-γ release assay (IGRA); obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing active tuberculosis and LTBI of the sample according to the comparison result.

In the present invention, the cut-off value may be selected by estimating a maximum likelihood through a likelihood ratio analysis value reflecting the prediction of sensitivity and specificity to predict an optimal diagnostic model.

In one embodiment, the method of simultaneously diagnosing active tuberculosis and LTBI according to the present invention may include: providing a whole blood (blood) sample of a patient diagnosed as positive in a tuberculin skin test (TST) or an interferon-γ release assay (IGRA); obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are higher than the cut-off value, and one or more selected from the group consisting of a hemoglobin level and a lymphocyte count are lower than the cut-off value as active tuberculosis.

In another embodiment, the method of simultaneously diagnosing active tuberculosis and LTBI may include: providing a whole blood (blood) sample of a patient diagnosed as positive in a tuberculin skin test (TST) or an interferon-γ release assay (IGRA); obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are lower than the cut-off value, and one or more selected from the group consisting of a hemoglobin level and a lymphocyte count are higher than the cut-off value as LTBI.

Still another aspect of the present invention provides a method of simultaneously diagnosing active tuberculosis and LTBI using one or more selected from the group consisting of a procalcitonin concentration, a C-reactive protein concentration, an α1-acid glycoprotein concentration and an erythrocyte sedimentation rate or a combination thereof as a biomarker.

In the present invention, the method of simultaneously diagnosing active tuberculosis and LTBI may use one or more selected from the group consisting of a procalcitonin concentration, a C-reactive protein concentration, an α1-acid glycoprotein concentration and an erythrocyte sedimentation rate of a specimen isolated from a human whole blood (blood) sample or a combination thereof as a biomarker.

Yet another aspect of the present invention provides a method of simultaneously diagnosing active tuberculosis and LTBI, which includes: providing a whole blood (blood) sample of a patient diagnosed as positive in a tuberculin skin test (TST) or an interferon-γ release assay (IGRA); obtaining one or more pieces of information selected from a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing active tuberculosis and LTBI of the sample according to the comparison result.

In the present invention, the cut-off value may be selected by estimating a maximum likelihood from a likelihood ratio analysis value reflecting the prediction of sensitivity and specificity to predict an optimal diagnostic model.

In one embodiment, the method of simultaneously diagnosing active tuberculosis and LTBI according to the present invention may include: providing a whole blood (blood) sample of a patient diagnosed as positive in a tuberculin skin test (TST) or an interferon-γ release assay (IGRA); obtaining one or more pieces of information selected from a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing a case in which one or more selected from the group consisting of a procalcitonin concentration, a C-reactive protein concentration, an α1-acid glycoprotein concentration and an erythrocyte sedimentation rate are higher than the cut-off value as active tuberculosis.

In another embodiment, the method of simultaneously diagnosing active tuberculosis and LTBI according to the present invention may include: providing a whole blood (blood) sample of a patient diagnosed as positive in a tuberculin skin test (TST) or an interferon-7 release assay (IGRA); obtaining one or more pieces of information selected from a procalcitonin concentration, a C-reactive protein concentration, an α1-acid glycoprotein concentration and an erythrocyte sedimentation rate from a specimen isolated from the whole blood (blood) sample; comparing the obtained information with a cut-off value; and diagnosing a case in which one or more selected from the group consisting of a procalcitonin concentration, a C-reactive protein concentration, an α1-acid glycoprotein concentration and an erythrocyte sedimentation rate are lower than the cut-off value as LTBI.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1A to 1D are sets of graphs that compare CBC analysis results among an active tuberculosis group, a latent tuberculosis infection (LTBI) group and a healthy control (A. White blood cell (WBC), B. Red blood cell (RBC), C. Platelet (PLT), D. Hemoglobin (Hb));

FIGS. 2A to 2D are sets of ROC curves based on the CBC analysis results between an active tuberculosis group and a healthy control (A. White blood cell (WBC), B. Red blood cell (RBC), C. Platelet (PLT), D. Hemoglobin (Hb));

FIGS. 3A to 3E are sets of graphs that compare diff count results among an active tuberculosis group, a LTBI group and a healthy control (A. Neutrophil, B. Lymphocyte, C. Monocyte, D. Eosinophil, E. Basophil);

FIG. 4 is a set of images showing the scatter plot of an automatic hematology analyzer among an active tuberculosis group, a LTBI group and a healthy control;

FIGS. 5A to 5E are sets of ROC curves based on a CBS diff count analysis result between an active tuberculosis group and a healthy control (A. Neutrophil, B. Lymphocyte, C. Monocyte, D. Eosinophil, E. Basophil);

FIGS. 6A to 6E are sets of graphs that compare analysis results for serum acute-phase proteins in an active tuberculosis group, an LTBI group and a healthy control (A. Endoglin (ENG), B. Procalcitonin (PCT), C. C-reactive protein (CRP), D. α1-acid glycoprotein (AGP), E. Erythrocyte-sedimentation rate (ESR)); and

FIGS. 7A to 7E are sets of ROC curves for acute-phase proteins between an active tuberculosis group and a healthy control (A. Endoglin (ENG), B. Procalcitonin (PCT), C. C-reactive protein (CRP), D. α1-acid glycoprotein (AGP), E. Erythrocyte-sedimentation rate (ESR)).

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the spirit and scope of the invention.

The present invention relates to a method of simultaneously diagnosing active tuberculosis and latent tuberculosis infection (LTBI) using one or more selected from the group consisting of a white blood cell count, a hemoglobin level, a neutrophil count, a lymphocyte count, monocyte count, a procalcitonin concentration, a C-reactive protein concentration, an α1-acid glycoprotein concentration and an erythrocyte sedimentation rate or a combination thereof as a biomarker.

Hereinafter, the present invention will be described in detail with reference to the accompanying drawings.

A study to differentiate an active tuberculosis group, a LTBI group and a non-infected (healthy) group using a serum protein is progressing. Since facilitating the collection of peripheral whole blood, this study is a method of diagnosing a disease by selecting a biomarker from serum or plasma. As MTB enters a host, a pathogenic mechanism between the MTB and the host is based on protein expression and protein-nucleic acid interactions. Such a tuberculosis-related protein may be used as a prognostic and diagnostic marker for identifying active tuberculosis.

A screening technique has rapidly evolved, and provides a wide-range platform for tuberculosis study and biomarker discovery. Particularly, the serum level of an acute-phase protein (APP) and increased cytokines discriminate tuberculosis patients. Tuberculosis is known to be associated with a change in serum level of an endogenous protein. An acute-phase reactive protein is produced in the liver in response to inflammation. A C-reactive protein is an acute-phase protein whose blood concentration increases to a high level in response to an interleukin-6 (IL-6)-mediated purulent infection such as active tuberculosis. Liver AGP production increases in acute inflammation as well as lung tuberculosis, and a similar result was found in tuberculosis patients having a specific glycosylated pattern of serum AGP, which may be useful in differential diagnosis of bacteria.

One aspect of the present invention provides a method of simultaneously diagnosing active tuberculosis and LTBI using one or more selected from a white blood cell count, a hemoglobin level, a neutrophil count, a lymphocyte count, a monocyte count, a procalcitonin concentration, a C-reactive protein concentration, an α1-acid glycoprotein concentration and an erythrocyte sedimentation rate of a specimen isolated from a human whole blood (blood) sample or a combination thereof as a biomarker.

Another aspect of the present invention provides a method of simply diagnosing whether a patient diagnosed as positive in a tuberculin skin test (TST) or an interferon-γ release assay (IGRA) is infected by active tuberculosis or LTBI, the method including the following steps:

providing a whole blood (blood) sample of a patient diagnosed as positive in a tuberculin skin test (TST) or interferon-γ release assay (IGRA);

obtaining one or more pieces of information selected from the group consisting of a white blood cell count, a hemoglobin level, a neutrophil count, a lymphocyte count, a monocyte count, a procalcitonin concentration, a C-reactive protein concentration, α1-acid glycoprotein concentration and an erythrocyte sedimentation rate from the specimen isolated from the whole blood (blood) sample;

comparing the obtained information with a cut-off value; and

diagnosing active tuberculosis and LTBI of the sample according to the comparison result.

In one embodiment, the cut-off value may be determined by a method of selecting a cut-off value by estimating a maximum likelihood through a likelihood ratio analysis value reflecting the prediction of sensitivity and specificity to predict an optimal diagnostic model.

Still another aspect of the present invention provides a diagnostic method including the following steps:

providing a whole blood (blood) sample of a patient diagnosed as positive in a tuberculin skin test (TST) or an interferon-γ release assay (IGRA);

obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample;

comparing the obtained information with a cut-off value; and

diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are higher than the cut-off value, and one or more selected from the group consisting of a hemoglobin level and a lymphocyte count are lower than the cut-off value as active tuberculosis.

In addition, the diagnostic method may include the following steps:

providing a whole blood (blood) sample of a patient diagnosed as positive in a tuberculin skin test (TST) or an interferon-γ release assay (IGRA);

obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample; and

comparing the obtained information with a cut-off value; and diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are lower than the cut-off value, and one or more selected from the group consisting of a hemoglobin level and a lymphocyte count are higher than the cut-off value as LTBI.

Yet another aspect of the present invention provides a method of simultaneously diagnosing active tuberculosis and LTBI using one or more selected from the group consisting of a procalcitonin concentration, a C-reactive protein concentration, an α1-acid glycoprotein concentration and an erythrocyte sedimentation rate or a combination thereof as biomarkers, the method including the following steps:

providing a whole blood (blood) sample of a patient diagnosed as positive in a tuberculin skin test (TST) or an interferon-γ release assay (IGRA);

obtaining one or more pieces of information selected from a procalcitonin concentration, a C-reactive protein concentration, an α1-acid glycoprotein concentration and an erythrocyte sedimentation rate from a specimen isolated from the whole blood (blood) sample;

comparing the obtained information with a cut-off value; and

diagnosing active tuberculosis and LTBI of the sample according to the comparison result.

In addition, the diagnostic method may include the following steps:

providing a whole blood (blood) sample of a patient diagnosed as positive in a tuberculin skin test (TST) or an interferon-γ release assay (IGRA);

obtaining one or more pieces of information selected from a procalcitonin concentration, a C-reactive protein concentration, an α1-acid glycoprotein concentration and an erythrocyte sedimentation rate from a specimen isolated from the whole blood (blood) sample;

comparing the obtained information with a cut-off value; and

diagnosing a case in which one or more selected from the group consisting of a procalcitonin concentration, a C-reactive protein concentration, an α1-acid glycoprotein concentration and an erythrocyte sedimentation rate are higher than the cut-off value as active tuberculosis.

Yet another aspect of the present invention relates to a diagnostic method including the following steps:

providing a whole blood (blood) sample of a patient diagnosed as positive in a tuberculin skin test (TST) or an interferon-γ release assay (IGRA);

obtaining one or more pieces of information selected from a procalcitonin concentration, a C-reactive protein concentration, an α1-acid glycoprotein concentration and an erythrocyte sedimentation rate from a specimen isolated from the whole blood (blood) sample;

comparing the obtained information with a cut-off value; and

diagnosing the one or more selected from the group consisting of a procalcitonin concentration, a C-reactive protein concentration, an α1-acid glycoprotein concentration and an erythrocyte sedimentation rate are lower than the cut-off value as LTBI.

Currently, tests for diagnosing active tuberculosis and LTBI are limited. To differentiate active tuberculosis from LTBI, it is necessary to develop an auxiliary biomarker, and effectively diagnose, treat and control tuberculosis. Therefore, to distinguish an active tuberculosis group, a LTBI group and a non-infected healthy control, a total of 126 preserved whole blood samples were subjected to CBC and diff count tests and ESR and multiplex bead arrays, targeting an acute-phase serum marker.

Hereinafter, the present invention will be described in detail with reference to the following examples. However, the following examples are merely provided to describe the present invention, and the present invention is not limited thereto.

### EXAMPLES

[Experimental Subjects]

The average age of an active tuberculosis group was 55.2 years old (23 to 89 years old, n=22), and a male-to-female ratio was 15:7 (68.2%:31.8%).

The average age of a LTBI group was 44.6 years old (21 to 70 years old, n=29), and a male-to-female ratio was 6:23 (20.7%:79.3%).

The average age of a healthy control was 33.2 years old (22 to 61 years old, n=58), and a male-to-female ratio is 12:46 (20.7%:79.3%).

Acid fast bacilli (AFB) staining results of the active tuberculosis group were 1+(n=2, 9.1%), 2+(n=4, 18.2%), 3+(n=4, 18.2%), 4+(n=4, 18.2%) and negative (n=8, 36.4%), and tuberculosis bacteria culture results were positive (n=19, 86.4%) and negative (n=3, 13.6%).

Results of a tuberculosis PCR test (nucleic acid amplification test) were positive (n=21, 95.5%) and negative (n=1, 4.5%).

Chest X-ray (CXR) results were positive (n=22, 100.0%) in the active tuberculosis group, positive (n=4, 13.8%) and negative (n=25, 86.2%) in the LTBI group, and negative (n=58, 100.0%) in the healthy control.

Results of an interferon-7 release assay (IGRA) were positive (n=29, 100.0%) in the LTBI group, and negative (n=58, 100.0%) in the healthy control.

TABLE 1

| Demographic and | | | | | Healthy | |
|---|---|---|---|---|---|---|
| clinical features | Active | | LTBI | | control | |
| Total number of | 22 | | 29 | | 58 | |
| subjects | | | | | | |
| Average age (range) | 55.2 | (23-89) | 44.6 | (21-70) | 33.2 | (22-61) |
| Sex, male/female | 15/7 | | 6/23 | | 12/46 | |
| AFB staining results | | | | | | |
| + positive, n(%) | 2 | (9.1) | NA | | NA | |
| ++ positive, n(%) | 4 | (18.2) | NA | | NA | |
| +++ positive, n(%) | 4 | (18.2) | NA | | NA | |
| ++++ positive, n(%) | 4 | (18.2) | NA | | NA | |
| − negative | 8 | (36.4) | NA | | NA | |
| TB bacterial culture | | | | | | |
| results | | | | | | |
| positive, n(%) | 19 | (86.4) | NA | | NA | |
| negative, n(%) | 3 | (13.6) | NA | | NA | |
| TB bacteria PCR | | | | | | |
| test results | | | | | | |
| positive, n(%) | 21 | (95.5) | NA | | NA | |
| negative, n(%) | 1 | (4.5) | NA | | NA | |
| CXR test results | | | | | | |
| positive, n(%) | 22 | (100.0) | 4 | (13.8) | 0 | (0) |
| negative, n(%) | 0 | (0) | 25 | (86.2) | 58 | (100.0) |
| IGRA results | | | | | | |

| | | | | | |
|---|---|---|---|---|---|
| positive, n(%) | NA | 29 | (100.0) | 0 | (0) |
| negative, n(%) | NA | 0 | (0) | 58 | (100.0) |

General features of groups participating in study showing number of subjects per group (n), average age, sex, AFB staining results, TB bacterial culture results, TB bacteria PCR test (nucleic acid amplification), CXR and IGRA results;
n: number;
negative: uninfected healthy group;
latent tuberculosis infection (LTBI) group;
Active tuberculosis: active pulmonary tuberculosis group;
NA: not applied.

### [Example 1] CBC Comparison Between Active Tuberculosis Group, LTBI Group and Healthy Control

According to complete blood cell count (CBC) analysis, the average total WBC counts in the active tuberculosis group, the LTBI group and the healthy control were 8.01±2.94 (1×10³/μL), 6.13±1.96 (1×10³/μL) and 5.70±1.41 (1×10³/μL), respectively.

Average RBC counts of the active tuberculosis group, the LTBI group and the healthy control were 4.14±0.40 (1×106/μL), 4.54±0.38 (1×106/μL) and 4.40±0.36 (1×106/μL), respectively.

Average platelet (PLT) counts in the active tuberculosis group, the LTBI group and the healthy control were 298.80±86.65 (1×10³/L), 276.72±58.56 (1×10³/L) and 257.30±48.57 (1×10³/μL), respectively.

Average hemoglobin (Hb) concentration values in the active tuberculosis group, the LTBI group and the healthy control were 12.10±1.66 (g/dL), 13.58±1.27 (g/dL) and 13.50±1.14 (g/dL), respectively.

Compared with the LTBI group and the normal control, the average WBC and PLT counts in the active tuberculosis group were considerably high, and the average RBC count and Hb concentration were considerably low.

The active tuberculosis group and the LTBI groups had statistically significant p values, for example, 0.0086 for the WBC count, 0.0006 for the RBC count and 0.0007 for the Hb concentration. There was no statistically significant difference in the WBC count, RBC count, Hb concentration or PLT count between the LTBI group and the healthy control. The active tuberculosis group and the healthy control had statistically significant p values, for example, less than 0.0001 for the WBC count, less than 0.0026 for the RBC count, less than 0.0001 for the Hb concentration, and 0.0084 for the PLT count. All of the three groups had statistically significant p values, for example, less than 0.0001 for the WBC group, less than 0.0008 for the RBC count, less than 0.0001 for the Hb concentration, and less than 0.0226 for the PLT count.

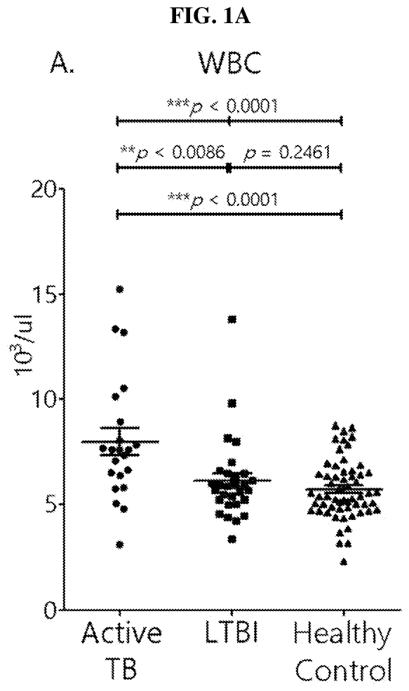

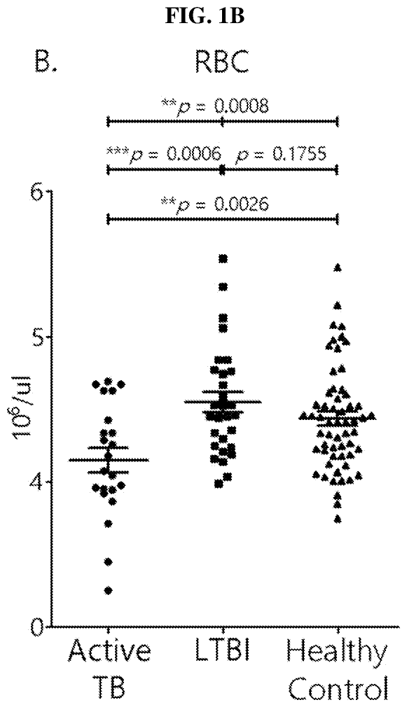

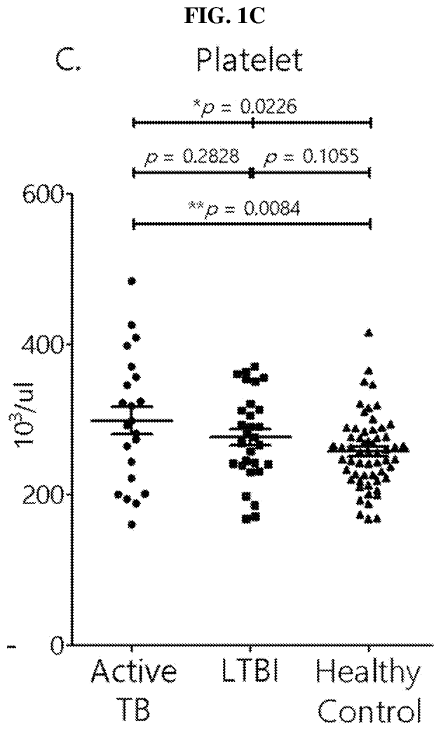

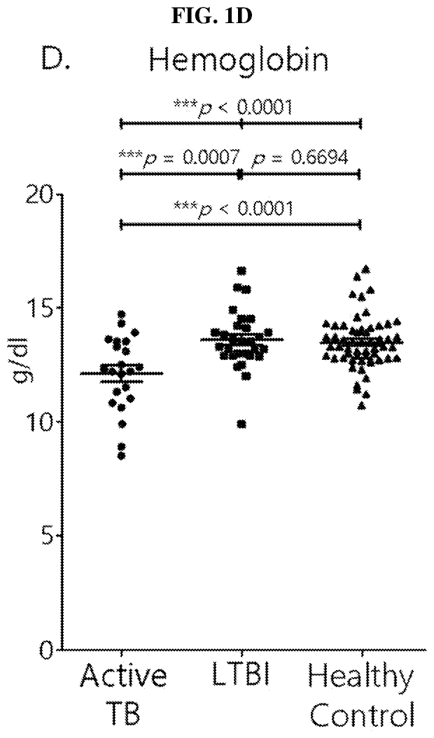

FIGS. 1A to 1D are sets of graphs comparing CBC analysis results between the active tuberculosis group, the LTBI group and the healthy control (A. white blood cell (WBC) count, B. red blood cell (RBC) count, C. hemoglobin (Hb) concentration, D. platelet (PLT) count).

The CBC results and statistic processing results between the active tuberculosis group, the LTBI group and the healthy control are listed in Tables 2 and 3.

TABLE 2

| | Active tuberculosis, | LTBI, | Healthy control, |
|---|---|---|---|
| | mean ± SD | mean ± SD | mean ± SD |
| Classification | (range) | (range) | (range) |
| WBC count(1 × 10³/μl) | 8.01 ± 2.94 | 6.13 ± 1.96 | 5.70 ± 1.41 |
| | (3.09-15.22) | (3.34-13.81) | (2.29-8.75) |
| RBC count(1 × 10⁶/μl) | 4.14 ± 0.40 | 4.54 ± 0.38 | 4.40 ± 0.36 |
| | (3.24-4.69) | (3.98-5.54) | (3.74-5.48) |
| PTL count (1 × 10³/μl) | 298.80 ± 86.65 | 276.72 ± 58.56 | 257.30 ± 48.57 |
| | 86.65(160.00-484.00) | (168.00-371.00) | (168.00-416.00) |
| Hb concentration (g/dL) | 12.10 ± 1.66 | 13.58 ± 1.27 | 13.50 ± 1.14 |
| | (8.50-14.70) | (9.90-16.60) | (10.70-16.70) |

TABLE 3

| | | | | Active |
|---|---|---|---|---|
| | | | Active | tuberculosis |
| | Active | LTBI vs. | tuberculosis | vs. LTBI vs. |
| | tuberculosis | healthy | vs. healthy | healthy |
| Classification | vs. LTBI | control | control | control |
| WBC count | 0.0086** | 0.2461 | <0.0001*** | <0.0001*** |
| RBC count | 0.0006*** | 0.1755 | 0.0026** | 0.0008*** |
| PTL count | 0.2828 | 0.1055 | 0.0084** | 0.0226* |
| Hb concentration | 0.0007*** | 0.6694 | <0.0001*** | <0.0001*** |

### [Example 2] ROC Curve Analysis Based on CBC Results

ROC curve analysis was performed to clinically apply the CBC analysis results. The p value of the ROC curve for the total WBC count was 0.0002, and the area under the curve (AUC) was 0.7727. The p value of the ROC curve for the RBC count was 0.0109, and the AUC was 0.6853. The p value of the ROC curve for the PLT count was 0.0396, and the AUC was 0.6497. The p value of the ROC curve for the Hb concentration was 0.0010, and the AUC was 0.7394. Among the CBC analysis results, the p values of the WBC, RBC and PLT counts and the Hb concentration were statistically significant (p<0.0500), and the AUC was approximately 0.7117.

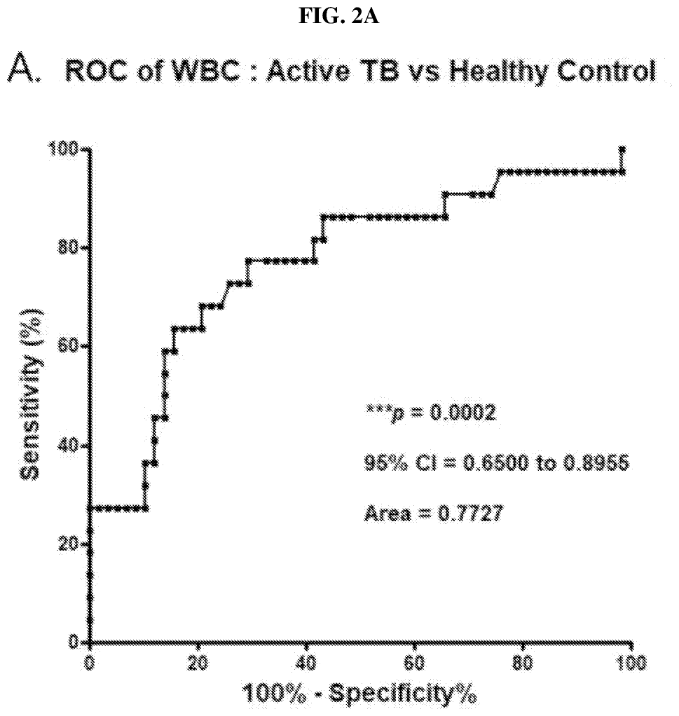

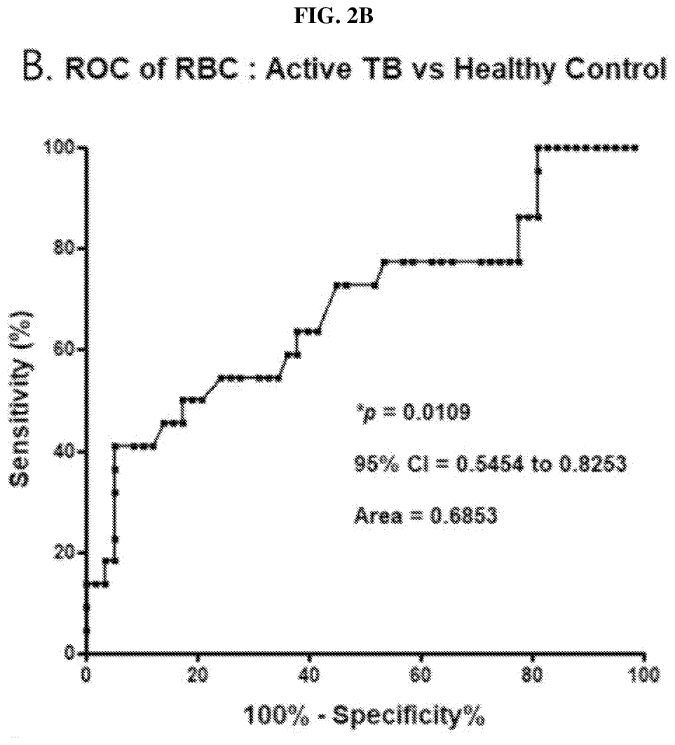

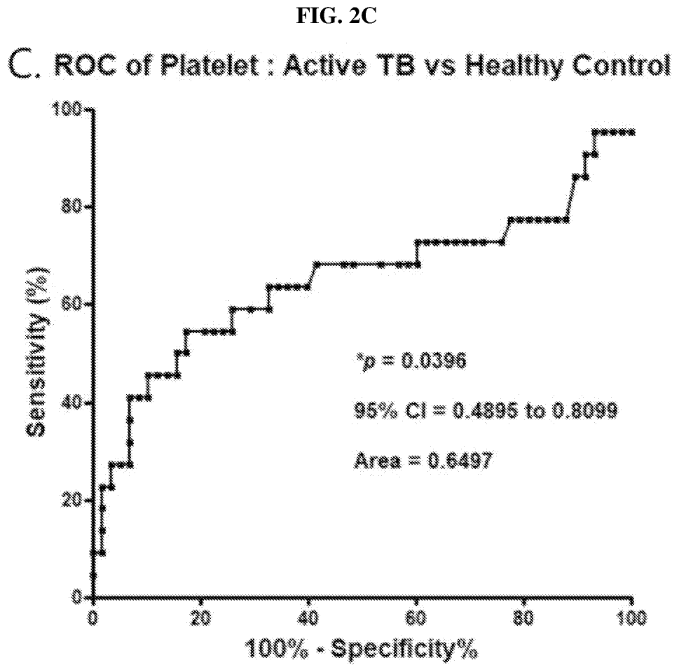

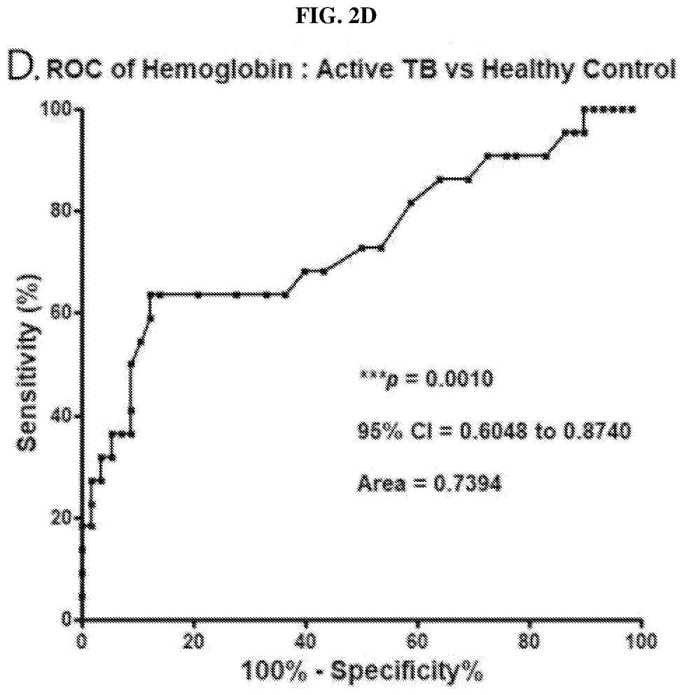

FIGS. 2A to 2D are sets of ROC curves based on the CBC analysis result between the active tuberculosis group and the healthy control (A. WBC count, B. RBC count, C. PLT count, and D. Hb concentration).

### [Example 3] Comparison of CBC Diff Counts Between Active Tuberculosis Group, LTBI Group and Healthy Control

Based on WBC diff count analysis, the average neutrophil counts in the active tuberculosis group, the LTBI group and the healthy control were 5.77±2.76×10³/μL (70.00%), 3.44±1.62×10³/μL (54.80%) and 3.25±1.21×10³/μL (55.61%).

The average lymphocyte counts in the active tuberculosis group, the LTBI group and the healthy control were 1.45±0.79×10³/μL (19.31%), 2.06±0.57×10³/μL (34.75%) and 1.91±0.44×10³/μL (34.73%).

The average monocyte counts in the active tuberculosis group, the LTBI group and the healthy control were 0.65±0.26×10³/μL (8.70%), 0.42±0.15×10³/μL (6.94%) and 0.38±0.10×10³/μL (6.80% each).

The average eosinophil counts in the active tuberculosis group, the LTBI group and the healthy control were 0.11±0.08×10³/μL (1.53%), 0.16±0.16×10³/μL (2.74%) and 0.12±0.11×10³/μL (2.17%).

The average basophil counts in the active tuberculosis group, the LTBI group and the healthy control were 0.03±0.02×10³/μL (0.43%), 0.05±0.02×10³/μL (0.78%) and 0.04±0.02×10³/μL (0.69%).

Compared with the LTBI group and the healthy control, in the active tuberculosis group, the average neutrophil and monocyte counts were significantly high, and the average lymphocyte and basophil counts were considerably low.

In the active tuberculosis group and the LTBI group, p values were statistically significant, for example, 0.0005 for the neutrophil count, 0.0026 for the lymphocyte count, 0.002 for the monocyte count, and 0.0117 for the basophil count.

There were no significant differences in neutrophil, lymphocyte, monocyte, eosinophil and basophil counts between the LTBI group and the healthy control.

In the active tuberculosis group and the healthy control, p values were statistically significant, for example, less than 0.0001 for the neutrophil count, 0.0014 for the lymphocyte count, and less than 0.0001 for the monocyte count.

All of the active tuberculosis group, the LTBI group and the healthy control had p values, for example, less than 0.0001 for the neutrophil count, less than 0.0006 for the lymphocyte count, and less than 0.0001 for the monocyte count, and less than 0.0245 for the basophil count.

There was no significant difference in eosinophil count between the active tuberculosis group, the LTBI group and the healthy control.

In automatic hematology analysis results for the active tuberculosis group, the LTBI group and the healthy control using an automatic hematology analyzer. the scattering sizes of NEUT+BASO and the monocyte count were increased in the WDR scatter plot of the active tuberculosis group. The scattering size of the WBC count in the active tuberculosis group was also increased in the WNR scatter plot of the active tuberculosis group. In the WNR scatter plots of the LTBI group and the healthy control, the degrees of scattering of NEUT+BASO and the monocyte count were low, and the degree of scattering of the lymphocyte count is increased, compared with the WNR scatter plot of the active tuberculosis group. In the WDF scatter plot of the LTBI group and the healthy control, the scattering size of the WBC count was smaller than the WDF scatter plot of the active tuberculosis group.

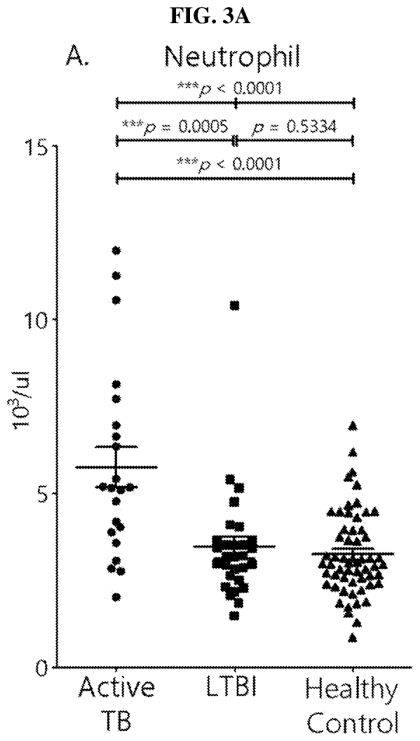

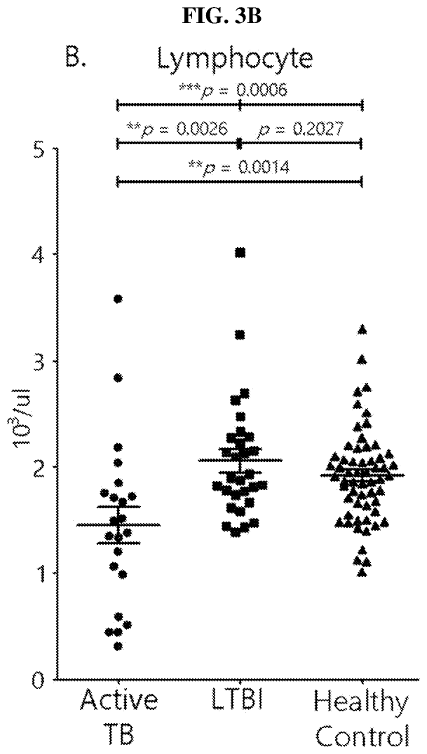

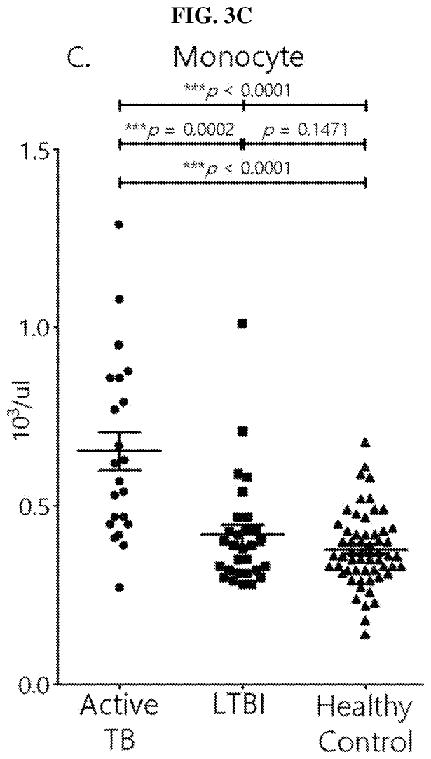

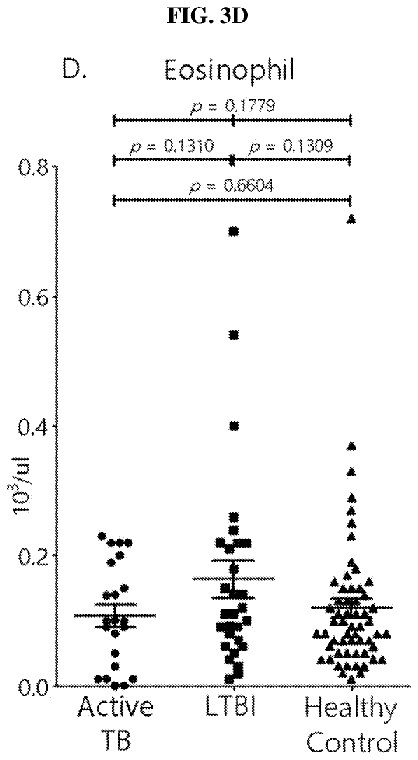

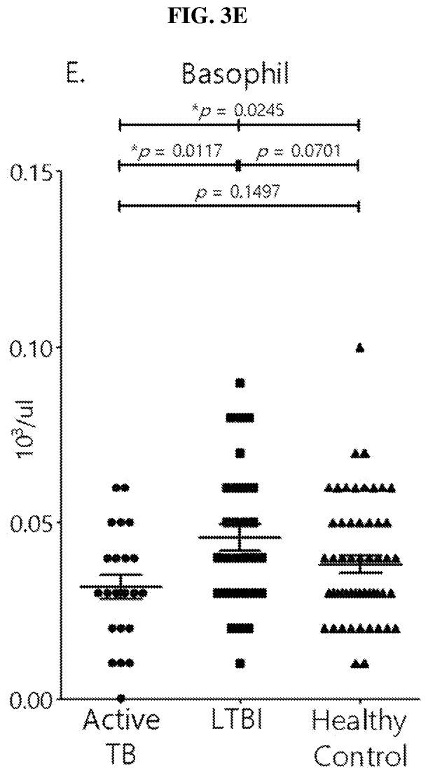

FIGS. 3A to 3E are sets of graphs that compare diff count results among the active tuberculosis group, the LTBI group and the healthy control (A. neutrophil count, B. lymphocyte count, C. monocyte count, D. eosinophil count, E. basophil count).

The CBC diff count test results and statistical processing results between the active tuberculosis group, the LTBI group and the healthy control are listed in Tables 4 and 5.

[Table 4]

TABLE 4

| | | Active tuberculosis, | | LTBI, average | | Healthy control, | |
|---|---|---|---|---|---|---|---|
| Cell type | | average (range) | | (range) | | average (range) | |
| Neutrophil | (1 × 10³/μl) | 5.77 ± 2.76 | (2.01-11.99) | 3.44 ± 1.62 | (1.48-10.40) | 3.25 ± 1.21 | (0.85-6.97) |
| count | % | 70.00 | (44.50-90.70) | 54.80 | (41.50-75.30) | 55.61 | (37.10-79.60) |
| Lymphocyte | (1 × 10³/μl) | 1.45 ± 0.79 | (0.31-3.58) | 2.06 ± 0.57 | (1.38-4.01) | 1.91 ± 0.44 | (1.01-3.29) |
| count | % | 19.31 | (4.90-44.60) | 34.75 | (16.10-50.00) | 34.73 | (12.70-50.10) |
| Monocyte | (1 × 10³/μl) | 0.65 ± 0.26 | (0.27-1.29) | 0.42 ± 0.15 | (0.28-1.01) | 0.38 ± 0.10 | (0.14-0.68) |
| count | % | 8.70 | (4.30-15.30) | 6.94 | (4.60-12.80) | 6.80 | (3.80-14.00) |
| Eosinophil | (1 × 10³/μl) | 0.11 ± 0.08 | (0.00-0.23) | 0.16 ± 0.16 | (0.01-0.70) | 0.12 ± 0.11 | (0.01-0.72) |
| count | % | 1.53 | (0.00-3.50) | 2.74 | (0.10-12.40) | 2.17 | (0.20-9.40) |
| Basophil | (1 × 10³/μl) | 0.03 ± 0.02 | (0.00-0.06) | 0.05 ± 0.02 | (0.01-0.09) | 0.04 ± 0.02 | (0.01-0.10) |
| count | % | 0.43 | (0.00-1.20) | 0.78 | (0.20-1.50) | 0.69 | (0.20-1.60) |

[Table 5]

TABLE 5

| | | | | Active tuberculosis |
|---|---|---|---|---|
| | Active tuberculosis | LTBI vs. healthy | Active tuberculosis | vs. LTBI vs. healthy |
| Cell type | vs. LTBI | control | vs. healthy control | control |
| Neutrophil count | 0.0005*** | 0.5334 | <0.0001*** | <0.0001*** |
| Lymphocyte | 0.0026** | 0.2027 | 0.0014** | 0.0006*** |
| Monocyte count | 0.0002*** | 0.1471 | <0.0001*** | <0.0001*** |
| Eosinophil count | 0.1310 | 0.1309 | 0.6604 | 0.1799 |
| Basophil count | 0.0117* | 0.0701 | 0.1497 | 0.0245* |

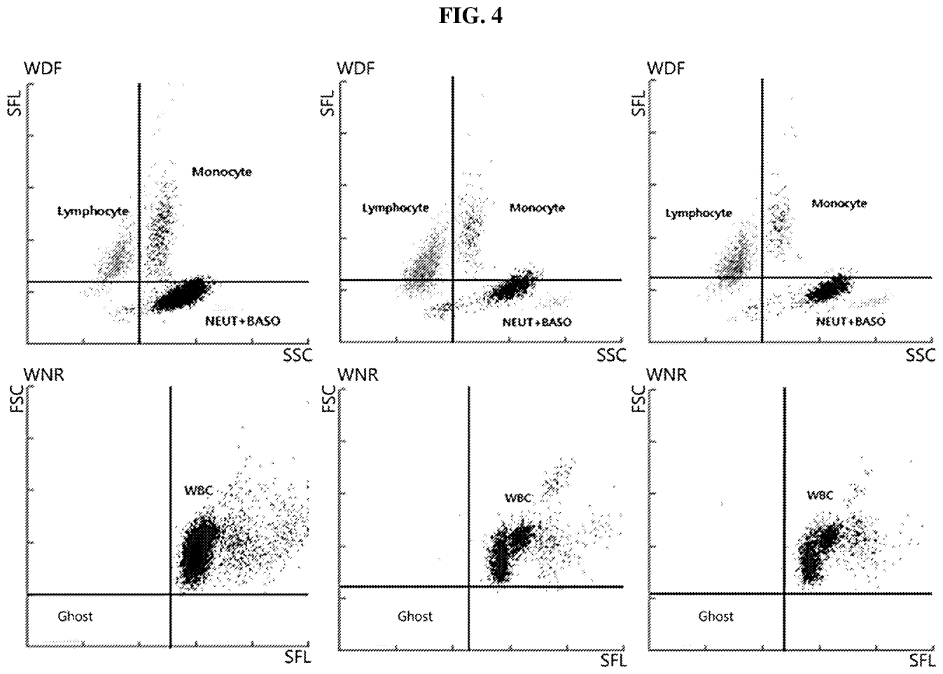

FIG. 4 is a set of images showing the scatter plot of automatic hematology analysis among the active tuberculosis group, the LTBI group and the healthy control (upper left: WDF in active tuberculosis group, lower left: WNR in active tuberculosis group, upper middle: WDF in LTBI group, lower middle: WNR in LTBI group, upper right: WDF in healthy control, lower right: WNR in healthy control).

### [Example 4] ROC Curve Analysis Based on CBC Diff Count Test Results

The analysis of an ROC curve was performed to clinically apply the results. The p value of the ROC curve for the neutrophil count was less than 0.0001, and the AUC was 0.8111. The p value of the ROC curve for the lymphocyte count was 0.0010, and the AUC was 0.7402. The p value of the ROC curve for the monocyte count was less than 0.0001, and the AUC was 0.8679. There was no statistically significant difference in the eosinophil or basophil count in the ROC curve. Among the WBC diff count test results, the p values of the neutrophil count, lymphocyte count and monocyte count were statistically significant (p<0.0500), and the AUC was approximately 0.8064.

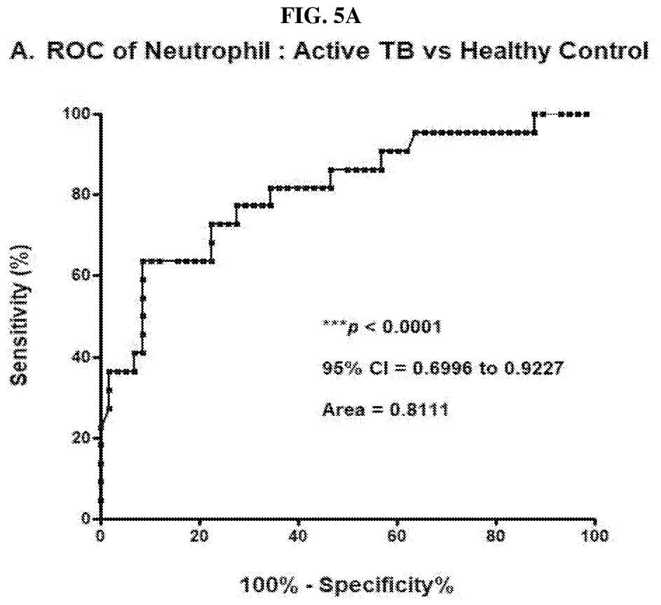

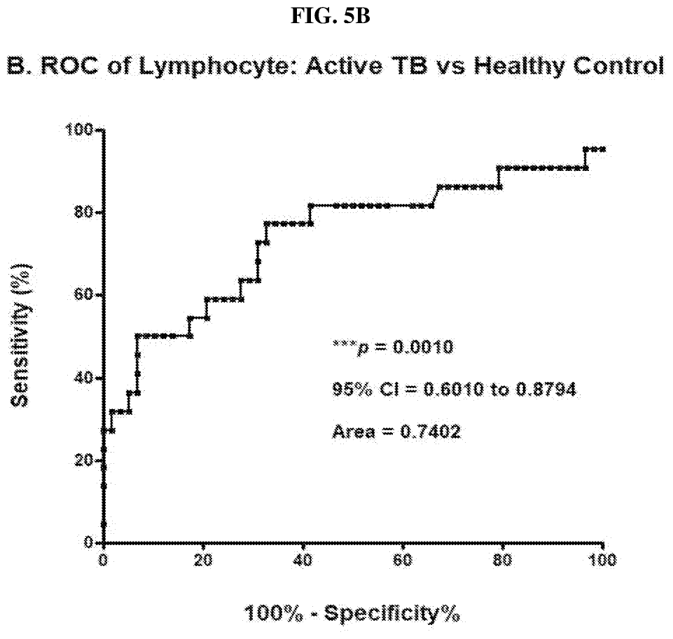

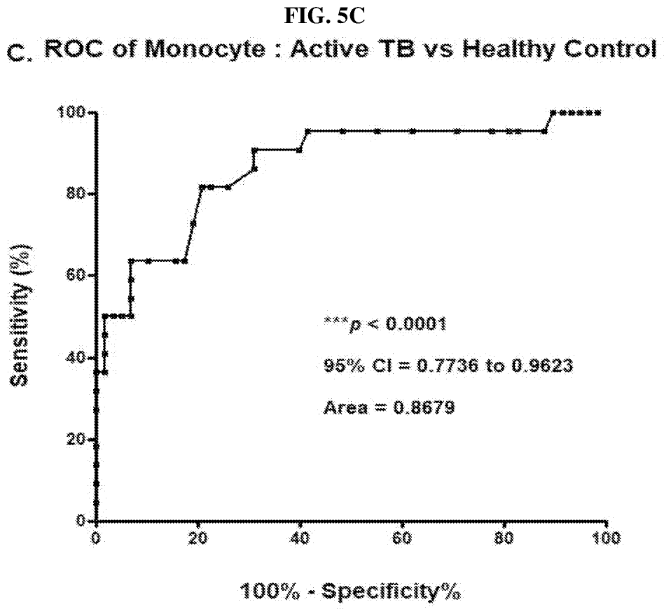

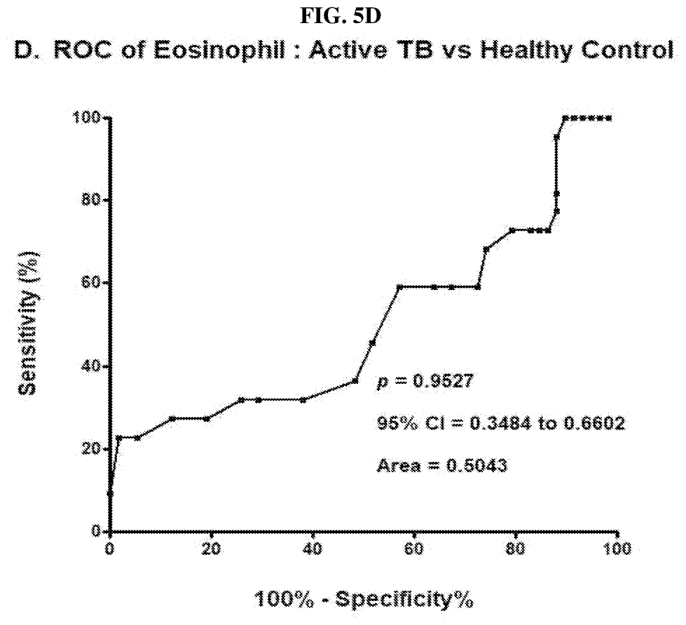

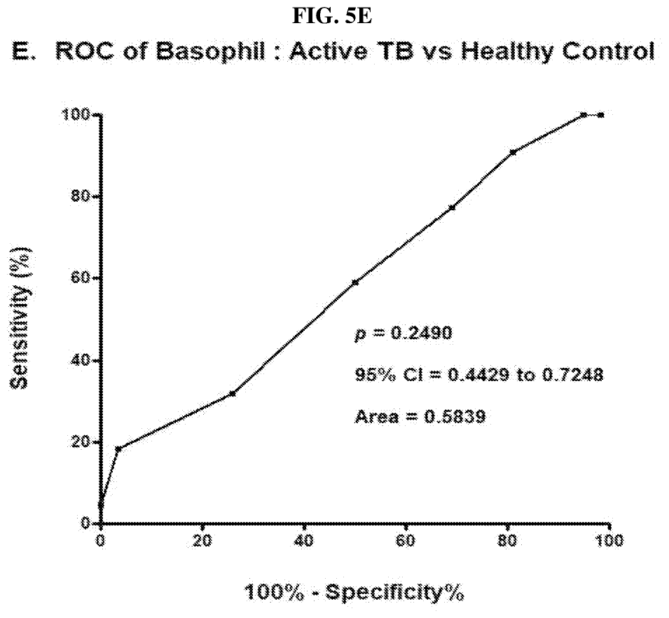

FIGS. 5A to 5E are sets of ROC curves based on a CBS diff count analysis result between the active tuberculosis group and the healthy control (A. neutrophil count, B. lymphocyte count, C. monocyte count, D. eosinophil count, E. basophil count).

### [Example 5] Comparison of Quantitative Analysis for Biomarkers of Acute-Phase Proteins in Active Tuberculosis Group, LTBI Group and Healthy Control

Based on the acute-phase protein analysis data, average endoglin (ENG) concentrations in the active tuberculosis group, the LTBI group and the healthy control were 1267.88±214.47 pg/mL, 1209.12±252.60 pg/mL and 1371.81±303.69 pg/mL, respectively.

Average PCT concentrations in the active tuberculosis group, the LTBI group and the healthy control were 44.11±29.21 pg/mL, 22.68±11.67 pg/mL and 18.15±4.58 pg/mL, respectively.

Average CRP concentrations in the active tuberculosis group, the LTBI group and the healthy control were 343491.91±362153.63 ng/mL, 2358.38±1213.21 ng/mL and 3375.52±1833.75 ng/mL, respectively.

Average α1-acid glycoprotein (AGP) concentrations in the active tuberculosis group, the LTBI group and the healthy control were 6886.68±2438.14 g/mL, 3749.57±1369.43 g/mL and 2969.90±795.71 g/mL, respectively.

Average erythrocyte sedimentation rates (ESRs) in the active tuberculosis group, the LTBI group and the healthy control were 59.59±34.19 mm/h, 13.90±11.45 mm/h and 4.0±2.5 mm/h, respectively.

Compared with the LTBI group and the healthy control, the average values of PCT, CRP and α1-acid glycoprotein (AGP) concentrations and ESRs in the active tuberculosis group were considerably high. Compared with the healthy control, the average values of the PCT, CRP and AGP concentrations and ESRs in the LTBI group were considerably high.

In the active tuberculosis group and the LTBI group, the p values were statistically significant, for example, 0.0007 for the PCT concentration, and less than 0.0001 for the CRP and AGP concentrations and the ESR. In the active tuberculosis group and the healthy control, the p values are statistically significant, for example, 0.0149 for the ENG concentration, 0.0112 for the PCT concentration, 0.0083 for the CRP concentration, 0.0012 for the AGP concentration, and less than 0.0001 for the ESR. In the active tuberculosis group, the LTBI group and the healthy control, the p values were statistically significant, for example, less than 0.0287 for the ENG concentration, and less than 0.0001 for the PCT, CRP and AGP concentrations and the ESR.

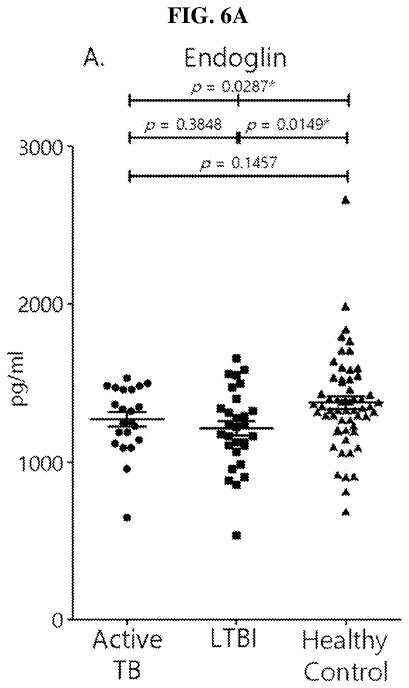

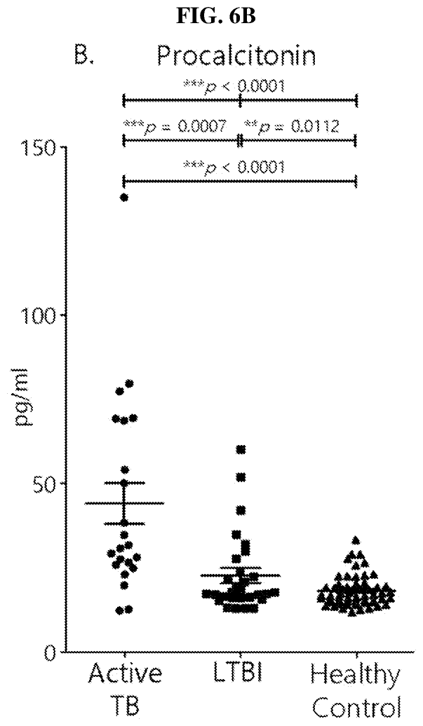

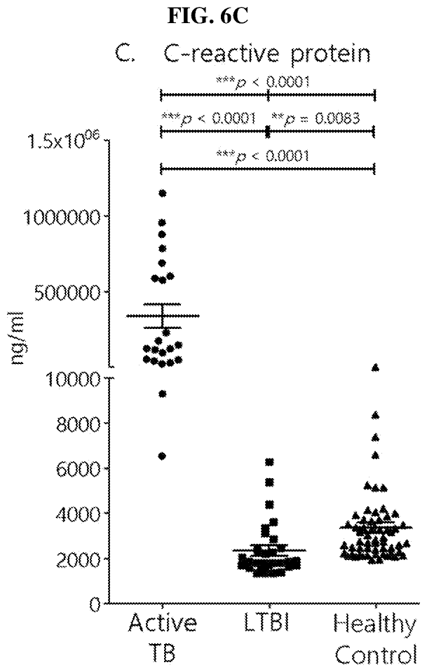

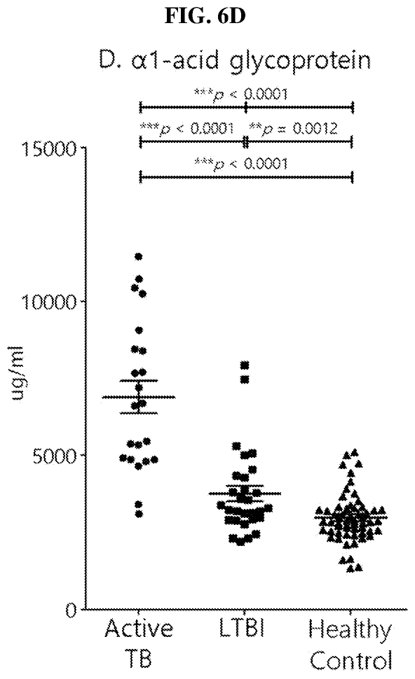

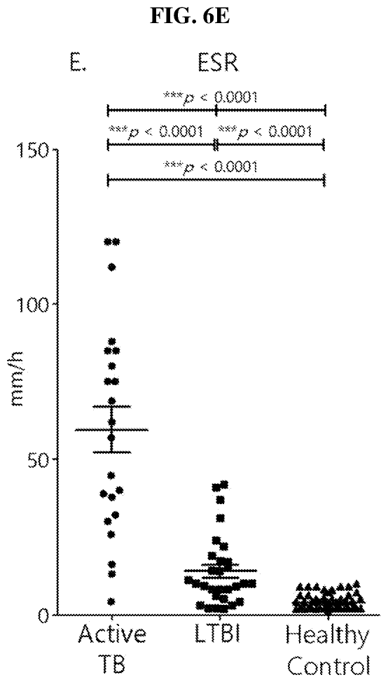

FIGS. 6A to 6E are sets of graphs that compare analysis results for serum acute-phase proteins in the active tuberculosis group, the LTBI group and the healthy control (A. ENG concentration, B. PCT concentration, C. CRP concentration, D. AGP concentration, E. ESR).

Serum acute-phase protein concentration analysis and statistical processing results for the active tuberculosis group, the LTBI group and the healthy control are listed in Tables 6 and 7 below.

TABLE 6

| Acute-phase protein | Active tuberculosis, | LTBI, average | Healthy control, |
|---|---|---|---|
| marker | average (range) | (range) | average (range) |
| ENG conc. (pg/mL) | 1267.88 ± 214.47 | 1209 ± 252.60 | 1371.81 ± 303.69 |
| | (643.50-1534.50) | (532.13-1658.25) | (680.63-2660.63) |
| PCT conc. (pg/mL) | 44.11 ± 29.21 | 22.68 ± 11.67 | 18.15 ± 4.58 |
| | (12.25-135.00) | (13.00-60.25) | (11.75-33.25) |
| CRP conc. (ng/mL) | 343491.91 ± 362153.63 | 2358.38 ± 1213.21 | 3375.52 ± 1833.75 |
| | (6552.00-1152788.00) | (1352.00-6266.00) | (1924.00-13000.00) |
| AGP conc. (ug/mL) | 6886.68 ±± 2438.14 | 3749.57 ± 1369.43 | 2969.90 ± 795.71 |
| | (3112.55-11445.53) | (2183.28-7932.83) | (1343.96-5117.25) |
| ESR (mm/h) | 59.59 ± 34.19 | 13.90 ± 11.45 | 4.00 ± 2.50 |
| | (4.00 ± 120.00) | (2.00-42.00) | (1.00-10.00) |

TABLE 7

| | | | Active | Active |
|---|---|---|---|---|
| Acute-phase | Active | LTBI vs. | tuberculosis | tuberculosis |
| protein | tuberculosis | Healthy | vs. healthy | vs. LTBI vs. |
| marker | vs. LTBI | control | control | healthy |
| ENG conc. | 0.3848 | 0.0149* | 0.1457 | 0.0287* |
| PCT conc. | 0.0007*** | 0.0112* | <0.0001*** | <0.0001*** |
| CRP conc. | <0.0001*** | 0.0083** | <0.0001*** | <0.0001*** |
| AGP conc. | <0.0001*** | 0.0012** | <0.0001*** | <0.0001*** |
| ESR | <0.0001*** | <0.0001*** | <0.0001*** | <0.0001*** |

### [Example 6] Analysis of ROC Curve for Acute-Phase Protein Concentrations

ROC curve analysis was performed to clinically apply the results. The p value of the ENG concentration was 0.1833, and the AUC was 0.5968. The p value of the PCT concentration was less than 0.0001, and the AUC was 0.8750. Thep value of the CRP concentration was 0.0001 or less, and the AUC was 0.9961. The p value of the AGP concentration was 0.0001 or less, and the AUC was 0.9671. Thep value of ESR was 0.0001 or less, and the AUC was 0.9820. Among the acute-phase protein concentration results, all of the p values of the PCT concentration, the CRP concentration, the AGP concentration and the ESR were significant (p<0.0500), and the AUC was approximately 0.9550.

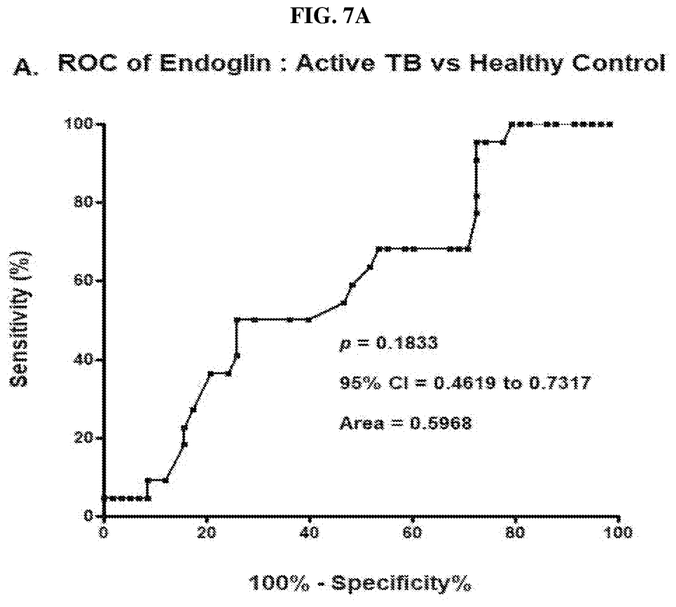

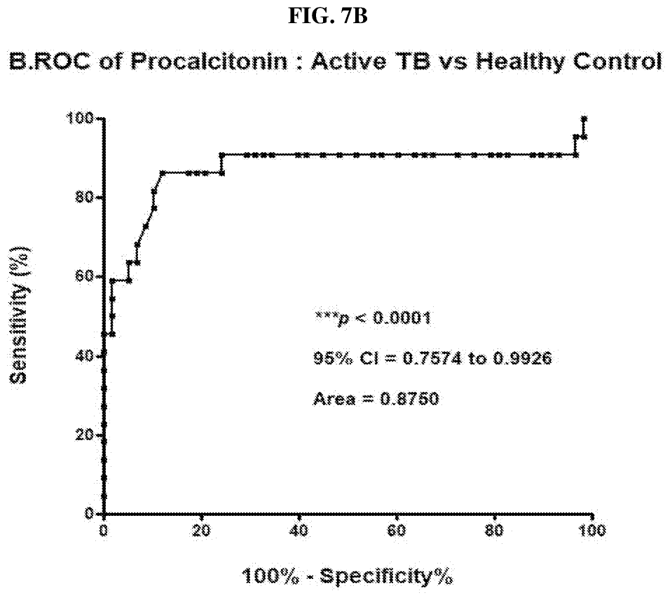

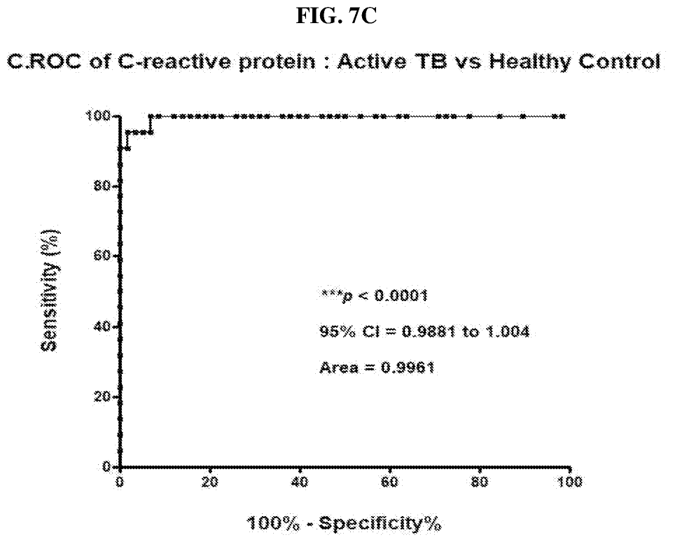

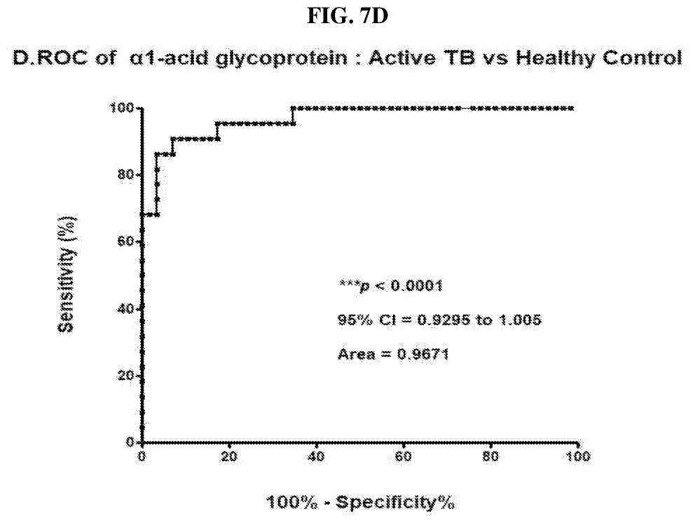

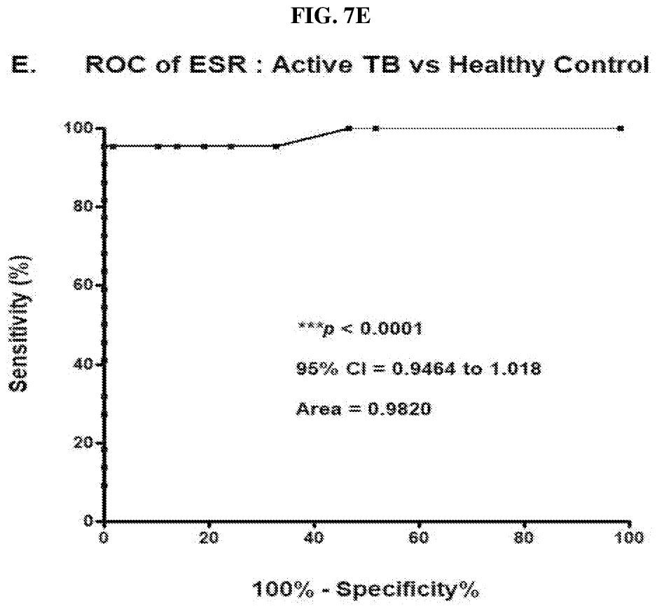

FIGS. 7A to 7E are sets of ROC curves for acute-phase proteins between the active tuberculosis group and the healthy control (A. ENG concentration, B. PCT concentration, C. CRP concentration, D. AGP concentration, E. ESR).

### [Example 7] Comparison of CBC and Diff Counts and Cut-Off Values of Acute-Phase Protein Markers to Distinguish Active Tuberculosis Group, LTBI Group and Healthy Control

The average WBC count for the active tuberculosis group included higher values, for example, n=16 (72.73%), and lower values, for example, n=6 (27.27%), compared with the cut-off value (6.49×10³/μL). The average WBC count for the LTBI group included higher values, for example, n=6 (20.69%), and lower values, for example, n=23 (79.31%), compared with the cut-off value (6.49×10³/μL). The average WBC count for the normal control included higher values, for example, n=15 (25.86%), and lower values, for example, n=43 (74.14%), compared with the cut-off value (6.49×10³/μL).

The average Hb concentration for the active tuberculosis group included higher values, for example, n=8 (36.36%), and the lower values, for example, n=14 (63.64%), compared with the cut-off value (12.55 g/dL). The average Hb concentration for the LTBI group included higher values, for example, n=25 (86.21%), and lower values, for example, n=4 (13.79%), compared with the cut-off value (12.55 g/dL). The average Hb concentration for the normal control included higher values, for example, n=51 (87.93%), and lower values, for example, n=7 (12.07%), compared with the cut-off value (12.55 g/dL).

The average neutrophil count for the active tuberculosis group included higher values, for example, n=16 (72.73%), and lower values, for example, n=6 (27.27%), compared with the cut-off value (4.01×10³/μL). The average neutrophil count for the LTBI value included higher values, for example, n=6 (20.69%), and lower values, for example, n=23 (79.31%), compared with the cut-off value (4.01×10³/μL). The average neutrophil count for the healthy control included higher values, for example, n=13 (22.41%), and lower values, for example, n=45 (77.59%), compared with the cut-off value (4.01×10³/μL).

The average lymphocyte count for the active tuberculosis group included higher values, for example, n=5 (22.73%), and lower values, for example, n=17 (77.27%), compared with the cut-off value (1.76×10³/μL). The average lymphocyte count for the LTBI included higher values, for example, n=21 (72.41%), and lower values, for example, n=8 (27.59%), compared with the cut-off value (1.76×10³/μL). The average lymphocyte count for the healthy control included higher values, for example, n=38 (66.52%), and lower values, for example, n=20 (34.48%), compared with the cut-off value (1.76×10³/μL).

The average monocyte count for the active tuberculosis group included higher values, for example, n=17 (77.27%), and lower values, for example, n=5 (2.73%), compared with the cut-off value (0.45×10³/μL). The average monocyte count for the LTBI group included higher values, for example, n=7 (24.14%), and lower values, for example, n=22 (75.86%), compared with the cut-off value (0.45×10³/μL). The average monocyte count for the healthy control included higher values, for example, n=12 (20.69%), and lower values, for example, n=46 (79.31%), compared with the cut-off value (0.45×10³/μL).

The average PCT concentration for the active tuberculosis group included higher values, for example, n=19 (86.36%), and lower values, for example, n=3 (13.64%), compared with the cut-off value (23.00 pg/m L). The average PCT concentration for the LTBI group included higher values, for example, n=8 (27.59%), and lower values, for example, n=21 (72.41%), compared with the cut-off value (23.00 pg/mL). The average PCT concentration for the healthy control included higher values, for example, n=7 (12.07%), and lower values, for example, n=51 (87.93%), compared with the cut-off value (23.00 pg/mL).

The average CRP for the active tuberculosis group included higher values, for example, n=21 (95.45%) and one lower value, for example, n=1 (4.55%), compared with the cut-off value (8853.00 ng/ml). The average CRP concentration for the LTBI group included no higher values, for example, n=0 (0.00%), and lower values, for example, n=29 (100.00%), compared with the cut-off value (8853.00 ng/ml). The average CRP concentration for the normal control included one higher value, for example, n=1 (1.72%), and lower values, for example, n=57 (98.28%), compared with the cut-off value (8853.00 ng/ml).

The average AGP concentration for the active tuberculosis group included higher values, for example, n=20 (90.91%), and lower values, for example, n=2 (9.09%), compared with the cut-off value (4548.00 μg/ml). The average AGP for the LTBI group included higher values, for example, n=5 (17.24%), and lower values, for example, n=24 (82.76%), compared with the cut-off value (4548.00 μg/ml). The average AGP for the healthy control included higher values, for example, n=4 (6.90%), and lower values, for example, n=54 (93.10%), compared with the cut-off value (4548.00 μg/ml).

The average ESR for the active tuberculosis group included higher values, for example, n=21 (95.45%), and one lower value, for example, n=1 (4.55%), compared with the cut-off value (11.50 mm/h). The average ESR for the LTBI group included higher values, for example, n=12 (41.38%), and lower values, for example, n=17 (58.62%), compared with the cut-off value (11.50 mm/h). The average ESR for the normal control included no higher values, for example, n=0 (0.00%), and lower values, for example, n=58 (100.00%), compared with the cut-off value (11.50 mm/h).

In the active tuberculosis group, the WBC count, the neutrophil count, the monocyte count, the PCT concentration, the CRP concentration, the AGP concentration and the ESR were significantly higher, and the Hb concentration and the lymphocyte count were considerably lower than those in the LTBI group and the healthy control.

The CBC and diff counts, and the cut-off values of acute-phase proteins of the active tuberculosis group, the LTBI group and the healthy control are shown in Table 8.

TABLE 8

| | WBC | | | | Neutrophil | | Lymphocyte | | Monocyte | |
|---|---|---|---|---|---|---|---|---|---|---|
| | count | | Hb | | count | | count | | count | |
| | (1 × 10³/ | | conc. | | (1 × 10³ | | (1 × 10³ | | (1 × 10³/ | |
| | [symbol] | | (g/dL) | | [symbol] | | [symbol] | | [symbol] | |
| | (+) | (−) | (+) | (−) | (+) | (−) | (+) | (−) | (+) | (−) |
| Active | 72.73 | 27.27 | 36.36 | 63.64 | 72.73 | 27.27 | 22.73 | 77.27 | 77.27 | 22.73 |
| tuberculosis | (16) | (6) | (8) | (14) | (16) | (6) | (5) | (17) | (17) | (5) |
| [symbol] | | | | | | | | | | |
| Positive | 43.24 | | 9.52 | | 45.71 | | 7.81 | | 47.22 | |
| predicted | (16/37) | | (8/84) | | (16/35) | | (5/64) | | (17/36) | |
| value, | | | | | | | | | | |
| [symbol] | | | | | | | | | | |
| LTBI | 20.69 | 79.31 | 86.21 | 13.79 | 20.69 | 79.31 | 72.41 | 27.59 | 24.14 | 75.86 |
| n = 29, | (6) | (23) | (25) | (4) | (6) | (23) | (21) | (8) | (7) | (22) |
| %(n) | | | | | | | | | | |
| Negative | 31.94 | | 16.00 | | 31.08 | | 17.78 | | 30.14 | |
| predicted | (23/72) | | (4/25) | | (23/74) | | (8/45) | | (22/73) | |
| [symbol] | | | | | | | | | | |
| Healthy | 25.86 | 74.14 | 87.93 | 12.07 | 22.41 | 77.59 | 65.52 | 34.48 | 20.69 | 79.31 |
| control | (15) | (43) | (51) | (7) | (13) | (45) | (38) | (20) | (12) | (46) |
| n = 58, | | | | | | | | | | |
| %(n) | | | | | | | | | | |

| | | | | | |
|---|---|---|---|---|---|
| Negative | 59.72 | 28.00 | 60.81 | 44.44 | 63.01 |
| predicted | (43/72) | (7/25) | (45/74) | (20/45) | (46/73) |
| [symbol] | | | | | |
| Cut-off | 6.49 | 12.55 | 4.01 | 1.76 | 0.45 |
| Sensitivity | 72.73 | 63.64 | 72.73 | 77.27 | 77.27 |
| Specificity | 74.14 | 87.93 | 77.59 | 65.52 | 79.31 |

| | PCT | | CRP | | AGP | | | |
|---|---|---|---|---|---|---|---|---|
| | conc. | | conc. | | conc. | | ESR | |
| | (pg/mL) | | (ng/mL) | | (μg/mL) | | (mm/h) | |
| | (+) | (−) | (+) | (−) | (+) | (−) | (+) | (−) |
| Active | 86.36 | 13.64 | 95.45 | 4.55 | 90.91 | 9.09 | 95.45 | 4.55 |
| tuberculosis | (19) | (3) | (21) | (1) | (20) | (2) | (21) | (1) |
| [symbol] | | | | | | | | |
| Positive | 55.88 | | 95.45 | | 68.97 | | 63.64 | |
| predicted | (19/34) | | (21/22) | | (20/29) | | (21/33) | |
| value | | | | | | | | |
| [symbol] | | | | | | | | |
| LTBI | 27.59 | 72.41 | 0.00 | 100.0 | 17.24 | 82.76 | 41.38 | 58.62 |
| n = 29, | (8) | (21) | (0) | (29) | (5) | (24) | (12) | (17) |
| %(n) | | | | | | | | |
| Negative | 28.00 | | 33.33 | | 30.00 | | 22.37 | |
| predicted | (21/75) | | (29/87) | | (24/80) | | (17/76) | |
| [symbol] | | | | | | | | |
| Healthy | 12.07 | 87.93 | 1.72 | 98.28 | 6.90 | 93.10 | 0.00 | 100.00 |
| control | (7) | (51) | (1) | (57) | (4) | (54) | (0) | (58) |
| n = 58, | | | | | | | | |
| %(n) | | | | | | | | |

| | | | | |
|---|---|---|---|---|
| Negative | 68.00 | 65.52 | 67.50 | 76.32 |
| predicted | (51/75) | (57/87) | (54/80) | (58/76) |
| [symbol] | | | | |
| Cut-off | 23.00 | 8853.00 | 4548.00 | 11.50 |
| Sensitivity | 86.36 | 95.45 | 90.91 | 95.45 |
| Specificity | 87.93 | 98.28 | 93.10 | 100.00 |

[symbol]

indicates data missing or illegible when filed

According to the present invention, a diagnostic method for simultaneously differentiating active tuberculosis and latent tuberculosis infection (LTBI) without additional tests for patients diagnosed as positive through a conventional tuberculosis infection assay such as a tuberculin skin test (TST) or an interferon-γ release assay (IGRA) can be provided.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A method of simultaneously diagnosing active tuberculosis and latent tuberculosis infection (LTBI) using one or more selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate, or a combination thereof as a biomarker.

2. The method of claim 1, wherein the method uses one or more selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate of a specimen isolated from a human whole blood (blood) sample, or a combination thereof as a biomarker.

3. A method of simultaneously diagnosing active tuberculosis and latent tuberculosis infection (LTBI), comprising:
providing a whole blood (blood) sample of a patient diagnosed as positive in a tuberculin skin test (TST) or an interferon-γ release assay (IGRA);
obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing active tuberculosis and LTBI of the sample according to the comparison result.

4. The method of claim 3, wherein the cut-off value is selected by estimating a maximum likelihood through a likelihood ratio analysis value reflecting the prediction of sensitivity and specificity to predict an optimal diagnostic model.

5. The method of claim 3, which comprises:
providing a whole blood (blood) sample of a patient diagnosed as positive in a TST or an IGRA;
obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are higher than the cut-off value, and one or more selected from the group consisting of a hemoglobin level and a lymphocyte count are lower than the cut-off value as active tuberculosis.

6. The method of claim 3, which comprises:
providing a whole blood (blood) sample of a patient diagnosed as positive in a TST or an IGRA;
obtaining one or more pieces of information selected from a white blood cell count, a hemoglobin concentration, a neutrophil count, a lymphocyte count, a monocyte count, a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing a case in which one or more selected from the group consisting of a white blood cell count, a neutrophil count, a monocyte count, a procalcitonin concentration, a C-reactive protein concentration, an α1-acid glycoprotein concentration and an erythrocyte sedimentation rate are lower than the cut-off value, and one or more selected from the group consisting of a hemoglobin level and a lymphocyte count are higher than the cut-off value as LTBI.

7. A method of simultaneously diagnosing active tuberculosis and latent tuberculosis infection (LTBI), comprising:
providing a whole blood (blood) sample of a patient diagnosed as positive in a tuberculin skin test (TST) or an interferon-γ release assay (IGRA);
obtaining one or more pieces of information selected from a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing active tuberculosis and LTBI of the sample according to the comparison result.

8. The method of claim 7, wherein the cut-off value is selected by estimating a maximum likelihood from a likelihood ratio analysis value reflecting the prediction of sensitivity and specificity to predict an optimal diagnostic model.

9. The method of claim 7, which comprises:
providing a whole blood (blood) sample of a patient diagnosed as positive in a TST or an IGRA;
obtaining one or more pieces of information selected from a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing a case in which one or more selected from the group consisting of a procalcitonin concentration, a C-reactive protein concentration, an α1-acid glycoprotein concentration and an erythrocyte sedimentation rate are higher than the cut-off value as active tuberculosis.

10. The method of claim 7, which comprises:
providing a whole blood (blood) sample of a patient diagnosed as positive in a TST or an IGRA;
obtaining one or more pieces of information selected from a procalcitonin concentration, a C-reactive protein concentration, an α1-acid glycoprotein concentration and an erythrocyte sedimentation rate from a specimen isolated from the whole blood (blood) sample;
comparing the obtained information with a cut-off value; and
diagnosing a case in which one or more selected from the group consisting of a procalcitonin concentration, a C-reactive protein concentration, an α1-acid glycoprotein concentration and an erythrocyte sedimentation rate are lower than the cut-off value as LTBI.
